(19) United States
(12) Patent Application Publication
(10) Pub. No.: US 2011/0117172 A1
(43) Pub. Date: May 19, 2011

(54) CROSS-LINKED POLYMER MATRIX, IN PARTICULAR FOR ADMINISTERING ACTIVE SUBSTANCES

(75) Inventor: Achim Gopferich, Sinzing (DE)

(73) Assignee: UNIVERSITAT REGENSBURG, Regensburg (DE)

(21) Appl. No.: 12/936,259

(22) PCT Filed: Apr. 3, 2009
(86) PCT No.: PCT/EP09/54023

§ 371 Date: Dec. 28, 2010

(30) Foreign Application Priority Data
Apr. 3, 2008 (DE) 10 2008 016 998.6

(51) Int. Cl. A61K 9/14; A61K 47/36; A61K 31/734; A61P 17/02; A61P 27/02

(52) U.S. Cl. 424/428; 424/488; 424/422; 514/779; 514/54

(57) ABSTRACT

The subject is cross-linked polymer matrices, which are used as active substance supports and can be applied locally or parenterally in human or animal bodies. The cross-linked polymer matrices are in particular self-dissolving cross-linked polymer matrices.

The subject of the present invention is polymer matrices that are used as active substance supports and can be applied locally in human or animal bodies. In particular, the present invention relates to such polymer matrices that release an incorporated active substance in a delayed manner at the site of application in the body over a time period of several hours or days and, in doing so, dissolve in the body fluid, so that they no longer need to be removed.

Embedding active substances in a matrix is a recognized procedure for releasing them in the body over a prolonged time period. The prerequisite for this is a suitable network in order to delay or even totally prevent diffusion within the matrix. In order to ensure a release over several hours, hydrogels are often employed.

Hydrogels, made up of gel-forming polysaccharides, such as alginates, for example, are generally known and have been described numerous times in the literature.

Alginates are salts of alginic acid, which contain, among other things, guluronic acid as monomer.

The individual polymer chains can be cross-linked by using polyvalent cations, with the cations forming ionic bonds with the guluronic acid of various polymer chains.

For example, DE 10 2004 019 241 A1 describes injectable cross-linked and non-cross-linked alginates and the use thereof in medicine and in cosmetic surgery as fillers for volume filling and defect filling—for example, for padding of wrinkles. It is described that the injected alginate can be dissolved once again at the site of application if needed, by injecting another agent that displaces the cations from the bond with the polymer chains and thus breaks the cross-linking. The injected alginate as such is not self-dissolving, however.

DE 103 23 794 A1 relates to a method for producing large-format, in particular, relatively thick (for example, 1 cm or more) alginate-based molded bodies. These molded bodies have a high wet strength and can be cut into thin layers and employed as a cosmetic skin patch or medicinal wound patch. They are further employed for oral, buccal, or nasal applications.

Involved in this case are durable, only poorly soluble gels.

A method for the controlled cross-linking of alginates is described in DE 697 07 475 T2. It is proposed so as to control the properties thereof, such as viscosity, elasticity, strength, etc., by adjusting the content of cross-links in the alginate.

DE 37 21 163 A1 relates to a support material for ophthalmic drugs. Proposed as support material is an alginate gel. The gel formation, that is, the cross-linking, takes place in vivo in this case by separate application of the alginate and the cross-linking agent to the eye and allowing the gel to form on site on the eye.

WO 2007/135114 A1 describes microcapsules made of hydrogels, with a mixture consisting of an anionic polysaccharide, such as, for example, an alginate, and an oligosaccharide derivative of chitosan being employed as gel-forming polymer components. The capsules obtained may also be employed as active substance supports. A self-dissolution is not provided for.

In order for hydrogels, as active substance supports, to be able to release the active substance over a prolonged time period, they must be cross-linked, as also described above in the prior art. Otherwise, they would swell strongly in aqueous (physiological) medium and lose their barrier function; as a result, the active substance would be released within an extremely short time. A delayed, gradual release over hours, for example, is not ensured with non-cross-linked hydrogels.

Also known are collagen and gelatin gels that are covalently cross-linked with reactive substances, such as glutardialdehyde, in order to limit the swelling. The release from these gels takes place over several hours. Of course, the method of preparing such gels is only poorly suited for peptide-based protein active substances, because the latter might be damaged during the cross-linking. Moreover, such covalently cross-linked gels can be eroded to only a limited extent and thus remain at the site of application beyond the therapy and, as a result, must be removed in order to prevent side effects.

Known to the skilled practitioner are three possibilities for the bioerosion of hydrogels: chemical hydrolysis of covalent bonds, enzymatic degradation, and dissolution in a solvent, with the solvent (such as, for example, water) abolishing the interactions between the polymer chains.

In the case of covalently cross-linked gels, only a chemical or enzymatic degradation comes into consideration as mechanisms. Of course, conventional covalent bonds are normally quite stable toward simple chemical hydrolysis; coming into consideration as biocatalysts in the living organism are a number of enzymes that allow this reaction to proceed under physiological conditions as well. Covalently cross-linked hydrogels can thus be degraded in the living organism, although this process either takes a very long time or else it is linked to the presence of the corresponding enzymes at the site of application.

For the purposes of the present invention, the aforementioned cross-linked hydrogels are not suitable. The covalently cross-linked hydrogels are unsuitable, because, in accordance with the invention, on the one hand, the matrices should dissolve within a few hours, but, on the other hand, should also be applicable at readily accessible regions of the body, such as, for example, the surface of the eye, on which only a few enzymes are present.

Coming into consideration as a suitable erosion mechanism, therefore, is only a dissolution of the matrix. Normally, the term dissolution is understood to mean a gradual “degradation” of a solid following its contact with a solvent, with aqueous systems coming into consideration as solvents for the present invention on account of the preferable application. Of course, cross-linked gels do not dissolve under such conditions, but only swell. Swelling is understood to mean an increase in the distance between the individual molecules of the gel-forming agent (such as, for example, polymer chains) by inclusion of solvent molecules. In this process, gels, such as the aforementioned alginates, cannot dissolve in the conventional sense, because the molecules of the gel-forming agent are held together at nodal points. Nodal points are understood to mean sites in the gel at which the molecules of the gel-forming agent are held together by a cross-linking agent. In order to dissolve a cross-linked hydrogel, this cross-linking agent must be removed or inactivated so as to break apart the nodal points.

If the nodal point is viewed as a combination of divalent or polyvalent ligand (=cross-linking agent) and receptor (=binding site on the gel-forming agent), the following possibilities or combinations thereof are conceivable in order to inactivate a cross-linking agent and thus dissolve such a gel once again:

### a) Reduction of the Affinity of the Ligand for the Receptor by Means of a Second, External Receptor

In the cross-linked gel, the ligand binds to the receptors with a certain affinity and thus binds together several molecules of the gel-forming agent. Through addition of a second, soluble receptor (which need not be identical to the one fixed to the gel-forming agent), the ligand is released from its bond. The prerequisite for this is a higher affinity of the ligand for the soluble receptor than for the one fixed to the gel-forming agent.

### b) Reduction of the Affinity of the Ligand for the Receptor by Changing the Ligand

By means of an external switch, for example, a change of the pH value or of the temperature, the structure of the ligand changes in such a way that it loses its affinity for the receptor at the gel structure. As a result, the bond between ligand and receptor is broken.

### c) Reduction of the Affinity of the Ligand for the Receptor by Changing the Receptor

By means of an external switch, for example, a change of the pH value or the temperature, the structure of the receptor changes in such a way that the ligand loses its affinity for the receptor and, as a result, the bond between the ligand and receptor is broken.

### d) Replacement of the Ligand by a Monovalent Form

By adding a second ligand, which, of course, on account of its structure, can bind to only one receptor in each case (=monovalent), the polyvalent ligand is displaced from its bond in the cross-linked gel and the fixed receptors are blocked. A schematic illustration of the mentioned possibilities for inactivation is shown below:

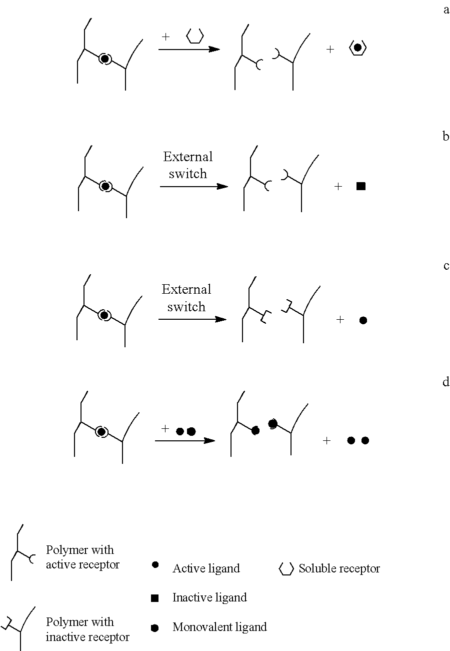

The present invention is thus based on the problem of providing a cross-linked polymer matrix that can be used as an active substance support with delayed active substance release. In particular, it was the problem to provide such a polymer matrix that dissolves by itself relatively rapidly, in particular within hours, at the site of application—for example, on or in the body of a human or animal.

According to one aspect of the invention, the dissolution is to take place independently of the amount of enzyme that is present at the site of application. In particular, it should also be possible to use the present invention for readily accessible regions of the body at which only a few enzymes are present, such as, for example, the surface of the eye.

According to another aspect, it was the problem of the present invention to provide a polymer matrix that can be employed as self-dissolving insert/implant for application in or on a body.

It was further a problem of the present invention to provide a polymer matrix or an insert produced from it that, as the active substance support, can release an active substance in the body over a prolonged period of time.

The above problem is solved by a polymer matrix according to claim 1, with the dependent claims relating to preferred embodiment features of the polymer matrix in accordance with the invention.

The polymer matrix according to the invention is based on a hydrogel constructed from a ternary system, the three components of which are a gel-forming polymer, a cross-linking agent, and a dissolving agent, the structure of which is illustrated schematically below:

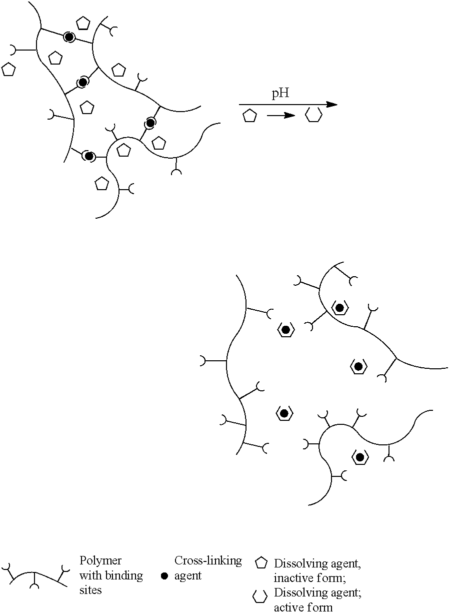

Polymers are long-chain macromolecules built of repeating units (so-called monomers). A polymer that is suitable for the present invention bears binding sites on its monomers for the cross-linking agent in regular or irregular sequence. On account of its structure, the latter can be bound by at least two such binding sites and consequently joins two or more polymer chains. In this way, the polymer and the cross-linking agent form the gel framework of the polymer matrix according to the invention.

The dissolving agent is distributed in it initially in an inactive form, without being fixed in place on one of the other components. In order to dissolve such a hydrogel according to the invention once again, the dissolving agent is activated by an external stimulus (for example, a change of the pH value). What is involved in the dissolution of the hydrogel according to the invention is accordingly a combination of the above-described possibilities a) and c).

In contrast to the above-described case a), the dissolving agent (the “soluble receptor” (above in a)) is not added, but rather is activated by an external stimulus, as described in possibility c). In contrast to the “receptor” in case c), the dissolving agent is freely mobile in the gel in accordance with the invention.

If the affinity of the dissolving agent either for the cross-linking agent or else for the binding site thereof on the polymer is changed by an external influence, the dissolving agent is bound either to the cross-linking agent or to the binding site thereof and, accordingly, a nodal point is broken apart.

Consequently, the gel framework is slowly destroyed and the individual components go into solution.

Examples of gel-forming polymers that are suitable for the present invention are polysaccharides that contain guluronic acid as one of their monomers. In these polysaccharides, the guluronic acid acts as a binding site for the cross-linking agent. To this end, the guluronic acid must be arranged along the polymer chain in blocks of at least two units in length. On account of their conformation, these blocks form the described binding site for the cross-linking agent.

An example of such guluronic acid-containing polysaccharides is alginates, which, besides the guluronic acid, additionally contain its isomer, mannuronic acid, as another monomer. According to a preferred embodiment of the invention, sodium alginate is used as the gel-forming polymer.

For the present invention, the content of guluronic acid in the gel-forming polymer, such as, for example, the alginate, should lie preferably between 30% (w/w) and 90% (w/w) and, in particular, between 35% (w/w) and 75% (w/w).

Examples of cross-linking agents that can bind to the guluronic acid units of such a polymer are polyvalent cations.

The latter are complexed by the guluronic acid blocks of various polysaccharide chains, so that a cross-linked gel framework is created.

Because of its low toxicity, Ca²⁺ is preferably employed in this process. The molar amount of calcium ions should lie between one-half and five times the polysaccharide content.

Depending on need and application, other cations, such as, for example, Fe^{2+/3+}, Al³⁺, Zn²⁺, or other cations known for this from the literature may also be employed. The molar amounts of cations to be used can be chosen in this case in accordance with the molar amount of calcium cations.

Dissolving agents in the sense of the invention, which can dissolve once again such a hydrogel cross-linked via polyvalent cations, are, for example, complex-forming agents that form stable complexes with polyvalent cations.

The stability of a complex is dependent, on the one hand, on the type of polyvalent cation and, on the other hand, on external influences, such as the existing pH value. Hence, the pH value can serve as an external stimulus, with the polyvalent cation being bound by the polymer at low pH values, for example, but being bound by the complex-forming agent at neutral to alkaline pH values.

An example of such a complex-forming agent is Na₂-EDTA (disodium ethylenediamine acetate¹), which is capable of releasing polyvalent cations, such as, for example, Ca²⁺ ions, from guluronic acid complexing. ¹Sic; tetraacetate?—Translator's Note

The dissolving agent should be contained in the polymer matrix in equimolar ratio to the cross-linking agent contained therein or to the binding sites for the latter.

However, for controlling the erosion or degradation rate, the molar amount of the dissolving agent in the polymer matrix may be varied, for example, by reducing it to one-half or increasing it threefold (in relation to the molar amount of cross-linking agent in each case).

The dissolving agent can be present in the polymer matrix in homogeneous distribution or, if need be, it can be distributed in selected regions. As a rule, a homogeneous distribution is preferred.

The type of active substance that can be incorporated into the polymer matrix is subject in principle to no limitations, as long as it is compatible with the polymer matrix.

In particular, the matrix according to the invention is suitable for the application of active substances, such as pharmaceuticals, that are a protein or peptide. Examples thereof are a growth factor, cytokine, epidermal growth factor, etc. It is also possible to employ active substances that form ionic interactions with the polymer matrix.

Shown are

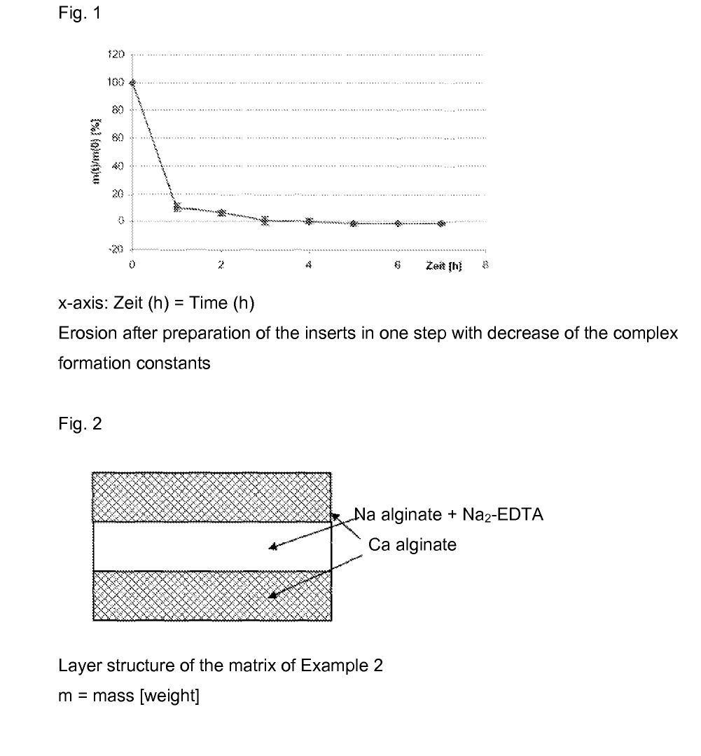

FIG. 1 a diagram with the dissolution behavior of the polymer matrix according to Example 1;

FIG. 2 the structure of a polymer matrix according to Example 2;

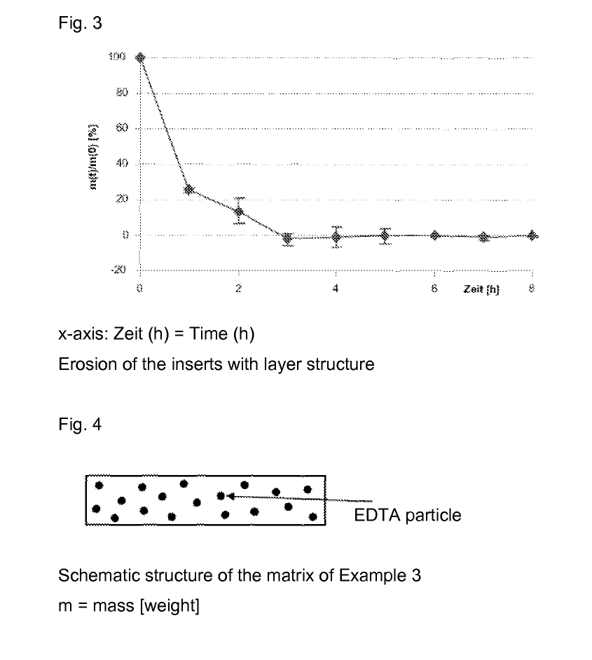

FIG. 3 a diagram of the dissolution behavior of the polymer matrix according to FIG. 2;

FIG. 4 the schematic structure of a polymer matrix according to Example 3; and

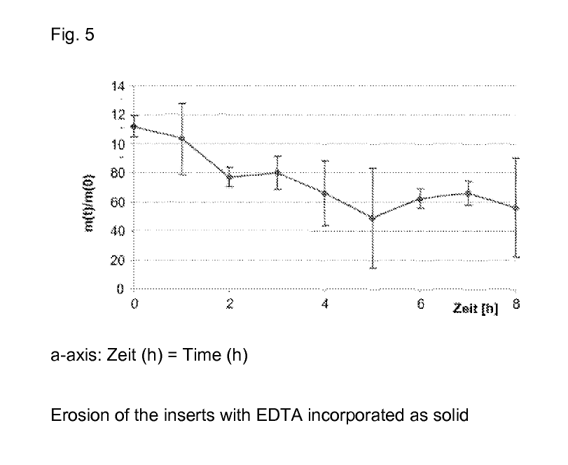

FIG. 5 the diagram of the dissolution behavior of the polymer matrix according to FIG. 4;

The present invention, the preparation thereof, and the application thereof will be explained below in detail, with reference, if necessary, to a preferred embodiment for better clarity, with the dissolving agent being a complex-forming agent, such as, in particular, EDTA, the cross-linking agent being calcium cation, and the gel-forming polymer being Na alginate.

Sodium alginate can readily be cross-linked even in the cold with Ca²⁺ ions, so that an active substance to be incorporated, such as, in particular, a protein/peptide, is not influenced in terms of its stability. To this end, the temperature can vary within a large range, it being limited downward by the freezing point of the corresponding solution; the limit upward is determined by the thermal stability of the active substance to be incorporated.

During the cross-linking, a Ca²⁺ ion is complexed by each of two successive guluronic acid units of two polysaccharide chains and, in this way, several chains are bound together. The cross-linked hydrogel that is formed is stable in form and has elastic properties, as in the case of a covalently cross-linked gel.

Two known methods for preparing a cross-linked alginate gel are described below:

1. Simple immersion of Na alginate gel, which is a non-cross-linked gel, in a Ca²⁺-containing solution, so that the Ca²⁺ ions can diffuse into the gel and displace the monovalent sodium ions from their bonding.

Of course, it is also possible to add the solution containing the polyvalent cation to the Na alginate gel.

2. By way of so-called “internal gel formation,” wherein the Ca²⁺ ions are released within the sodium alginate solution and the polysaccharide chains are thus cross-linked from inside to the outside. To this end, either a poorly soluble calcium salt or a complex-forming agent that delivers the Ca²⁺ ions only slowly is employed.

In order to dissolve once again a cross-linked calcium alginate gel, the Ca²⁺ ions must be removed and replaced by monovalent cations, for example, Na⁺ ions. That is most easily possible by simple diffusion of the monovalent cations, although this process normally takes a very long time and necessitates an excess of monovalent cations.

In order to accelerate the dissolution, the Ca²⁺ ions may be bound by complex-forming agents with elevated affinity toward Ca²⁺ ions, so that they are no longer available for cross-linking. The prerequisite for this is that the newly created complex is sufficiently stable to release the calcium ion from the bond on the guluronic acid blocks. This is normally the case for complex-forming agents that engage in very stable complexes with polyvalent cations (logarithmic complex formation constants in the range of about 7 to 36, depending on the cation).

A problem with this approach is that the cross-linked gel must be immersed in a solution of the complex-forming agent, which is ruled out for biological applications.

In accordance with the invention, this problem is solved by adding the complex-forming agent in inactive form as a dissolving agent to the cross-linked hydrogel, but making it possible to switch on its activity. In other words, the dissolving agent is already incorporated in inactive form in the polymer matrix according to the invention.

An example of such a “switchable” complex-forming agent is EDTA, which, at low pH values, forms stable complexes with monovalent cations, such as the sodium cation, but, at a neutral or basic pH value in the range of 7 and higher, such as, for example, that existing in bodily fluids, has a greater complex-forming affinity for polyvalent cations such as Ca²⁺.

During the preparation of the polymer matrix according to the invention, care must be taken that the complex-forming agent employed as dissolving agent does not hinder the cross-linking of the polymer chains by the polyvalent cations. The complex leading to dissolution of the matrix, that is, the activation of the dissolving agent, should thus be created only after application of the polymer matrix at the site of application.

This can, for example, occur by way of a spatial separation of complex-forming agent and cation during the preparation, which is then reversed once again through the bodily fluid present after the application, as in the embodiments according to Examples 2 and 3.

To this end, the complex-forming agent can be incorporated as an undissolved solid in the polymer matrix and can be present in the latter in a distributed manner. When it is in contact with a bodily fluid, the solid goes gradually into solution and the complex-forming agent begins to act.

The polymer matrix can also be structured as a layer system.

In this case, the spatial separation can be brought about in that the dissolving agent is added to only a part of the layers, whereas the rest of the layers contain no dissolving agent.

Example 2 shows a preferred embodiment of the spatial separation in a layer system, with the layer containing the dissolving agent being non-cross-linked and arranged between cross-linked layers.

Another, by far more elegant method is the temporary lowering of the stability of the complex made up of polyvalent cation and complex-forming agent, so that the ions can be bound more readily by the guluronic acid blocks. This lowering of the complex formation constant must also be reversible, this being ensured, for example, by utilization of EDTA as the complex-forming agent.

The preparation of such a polymer matrix can take place in analogy to the method of internal gel formation of alginates that is known from the literature. In this process, the pH value of a solution of gel-forming polymer is reduced by a hydrolysis-labile, acid-liberating agent. As a result of the low pH value, Ca²⁺ ions are released from the Ca-EDTA complex and lead to the cross-linking of the polymer.

In place of internal gel formation, it is also possible to utilize another procedure for cross-linking, such as, for example, the simple diffusion of Ca²⁺ ions out of a solution or a controlled diffusion through membranes, this listing not being exhaustive.

The gels obtained can be dried.

The obtained polymer matrices, which optionally have been dried if necessary, can be divided into pieces of sizes suitable for the respective application. The pieces that have optionally been brought to the desired size can be employed as inserts, which, depending on the active substances possibly contained in them, can be stored in a suitably packaged form and can be employed directly.

The term “insert” includes also an implant that is introduced into the body.

The polymer matrix according to the invention can also be produced in situ according to the above procedure, directly at the site of application in the body. To this end, the reaction conditions should preferably be chosen such that the cross-linking takes place rapidly, for example by choosing the pH value of the reaction solutions accordingly; in the case of EDTA, then, in the acid range.

For the in situ generation, the reaction solutions, as well as the non-cross-linked gel-forming polymer and the solution with cross-linking agent, can be applied in any way to the site at which the cross-linked polymer matrix is to be created, such as, for example, by injection, spraying, brushing, etc.

In the case of a dried matrix, it is rehydrated by bodily fluid (in the case of application on the eye, by tear fluid) during application in the human or animal body, whereby it swells slightly. At the physiological pH value of 7.4, the Na₂-EDTA incorporated in the matrix is “activated”; that is, the complex formation constant is increased, resulting in the creation of a more stable Ca-EDTA complex. The cross-linking Ca²⁺ ions are consequently withdrawn from the matrix, so that the polymer chains can slowly dissolve.

The swelling of the polymer network assists the diffusion of the complex-forming agent to the binding sites with the polyvalent cations, particularly also in the case of initial spatial separation, in order to complex the latter. In this way, the matrix dissolves in the bodily fluid surrounding it within a few hours and need no longer be removed in a tedious fashion following the application.

The polymer matrix according to the invention is characterized, in particular, by the fact that it already incorporates a dissolving agent. Following activation of the dissolving agent by an external stimulus, the dissolving agent brings about the breaking of the bond between binding site and cross-linking agent, so that the polymer matrix can dissolve.

The time duration of the dissolution can be adjusted and controlled, for example, by the type and/or amount of dissolving agent as well as the manner in which the agent is arranged in the matrix.

The polymer matrix according to the invention is thus suitable, in particular, for the preparation of inserts, which, after application in the human or animal body, dissolve by themselves or else are resorbed by the latter.

The polymer matrix according to the invention or the inserts obtained from it can be used for parenteral application, for example, in body cavities or else for the local administration of active substances.

An example of this is the local application on the eye. In this case, the activation of the dissolving agent can take place by means of the tear fluid.

Another example of a parenteral application of the polymer matrix according to the invention is its use as a membrane, in particular, as a barrier membrane, for reducing postoperative scarring following, for example, surgical interventions in or on the body.

Postoperative scarring between a surgical wound and the adjoining surrounding tissue, also referred as adhesion, is a serious medical problem in postoperative wound care.

In accordance with the invention, the term “postoperative scarring” encompasses any form of creation of scar tissue, such as, for example, adhesion, and formation of accretions.

Scarring or adhesion is a condition that comprises the formation of abnormal tissue connections. These tissue connections are detrimental to bodily functions, can obstruct the intestines and other parts of the gastrointestinal tract (intestinal obstruction), and can cause infertility and general discomfort, for example, pelvic pain. In the worst case, this condition can be life-threatening.

The most common form of scarring occurs following a surgical intervention, but it also occurs as a result of other inflammatory processes or event, such as mechanical injuries.

Known for the prevention or reduction of scarring is the spatial separation of freshly operated sites from adjoining surrounding tissue by application of so-called barrier membranes. As a result, cell migration and vascular ingrowths from the neighboring tissue into the freshly operated site are prevented or slowed, so that wound healing can proceed undisturbed. For example, following a surgical intervention in the body, a barrier membrane can be placed on the freshly operated region, which, then, after wound closure, shields the freshly operated site from overlying muscles.

EP 1 588 675 B1 describes an example of such a barrier membrane, with the barrier membrane being a biodegradable polylactide polymer. The membrane is prepared in vitro in this case, outside of the body, and is brought into appropriate form and, if need be, applied.

In contrast to this, the polymer matrix according to the invention can be obtained in situ directly in the region of the operated site. As described above, the reaction solutions for creating a cross-linked gel can be brought directly to the desired site for this purpose, for example, by injection, spraying, brushing, etc.

In particular, active substances that promote wound healing or assist and accelerate the healing in other ways may also be added to it simultaneously.

As described above, the polymer matrix according to the invention can be obtained in a thickness and shape that is appropriate for the respective application.

Suitable thicknesses and shapes for utilization as a barrier membrane can be taken, for example, from the aforementioned EP 1 588 675 B1.

For example, suitable thicknesses lie in a range of between 10 μm and 300 μm and preferably between 10 and 100 μm.

For the use as a barrier membrane, the dissolution time period should lie preferably between 7 days and 12 weeks. As already discussed, the dissolution time period can be adjusted in diverse ways, for example, by the type of dissolving agent, the quantity of dissolving agent, its distribution in the membrane, etc.

In particular, membranes having a dissolution time period of longer than 30 days can be employed also as a soft tissue support.

Examples for the preparation of the described matrices

### EXAMPLE 1

### Reduction of the Complex Formation Constant

The matrix was prepared from Na alginate (65-75% guluronic acid) in one step by means of internal cross-linking. To this end, CaCl₂.2H₂O and Na₂EDTA.2H₂O in equimolar ratio were initially dissolved in bidistilled water and the pH value of the solution was adjusted to 7-7.5 using sodium hydroxide. At this pH value, the Ca-EDTA complex is most stable. The sodium alginate was mixed with glycerin and then dissolved in the Ca-EDTA solution (2% (w/v) alginate, 5% (w/v) glycerin). To this solution was added 1% (w/v) glucono-δ-lactone, which undergoes hydrolysis slowly in aqueous solution to gluconic acid and thereby lowers the pH value, which makes the Ca-EDTA less stable.

In order to prevent the premature hydrolysis to gluconic acid, the solution was sterile-filtered immediately after addition of the lactone and poured into a Teflon dish, covered, and allowed to stand until the gel had formed completely.

Afterwards, the gel was dried overnight at room temperature and subsequently eye inserts of 5-mm diameter were punched out.

For checking the erosion, these inserts were incubated in 5 mL of Tris buffer (pH 7.4) at 37° C.; each hour, the condition of the inserts was inspected macroscopically and the supernatant was pipetted off. After drying the residue, it was weighed (n=3) in order to be able to display graphically the dissolution of the inserts.

The test was carried out over 8 hours.

The result was that a degradation by approximately 90% took place within the first hour, with complete dissolution after approximately three hours (see FIG. 1).

### EXAMPLE 2

### Spatial Separation of Cations and EDTA by Layerwise Buildup of the Matrix

For spatial separation of complex-forming agents and cations, the matrix was built up from three layers (see FIG. 2). Initially, CaHPO₄.2H₂O was suspended in neutral buffer and the Na alginate was dissolved in this suspension. Following addition of glucono-δ-lactone, sterile filtration was carried out and the first layer was first allowed to gel in a Teflon dish and then allowed to dry. Afterwards, a sterile-filtered Na alginate solution, which contained Na₂EDTA, was added on top of the dried first layer and allowed to dry without cross-linking. The uppermost layer was sterile-filtered in analogy to the first layer and applied.

Once the matrix was completely dry, eye inserts of 5-mm diameter were punched out and investigated in terms of their erosion (see FIG. 3).

The two outer, cross-linked layers prevent the immediate dissolution of the matrix. Only after swelling has occurred can these two layers dissolve, as soon as the free Na₂EDTA from the middle layer complexes the Ca²⁺ ions of the two outer layers.

It was found as a result that complete dissolution, as in Example 1, likewise had taken place after approximately hours,* but the degradation after one hour was only about 75%. *sic; Translator's note.

### EXAMPLE 3

### Spatial Separation of Cations and EDTA by Utilization as a Solid

These matrices were composed of only one layer. The complex-forming agent was incorporated in this case as a solid (see FIG. 4) and dissolves slowly only after contact with the bodily fluid. Initially, an EDTA suspension was prepared, in which a Na alginate having a low guluronic acid fraction (35-45%) was dissolved. This viscous emulsion was drawn out to form a film, dried overnight, and subsequently cross-linked by placing it for one minute in a 5% CaCl₂ solution. Once the film had dried again completely, eye inserts of 5-mm diameter were punched out.

The erosion test was carried out in analogy to the others. Found in the case of this matrix structure, however, was that the inserts dissolved only very slowly and, after 8 hours, had not yet completely dissolved (see FIG. 5). Such a structure of the described matrix is suitable accordingly for a long-term application, in particular.

1. A polymer matrix, wherein the polymer matrix is formed from gel-forming polymers and a cross-linking agent, wherein the gel-forming polymers have binding sites that react with the cross-linking agent to form cross-links, wherein the matrix additionally contains a dissolving agent, which, after activation, breaks once again the cross-linking and brings about the self-dissolution of the polymer matrix.

2. The polymer matrix according to claim 1,
wherein the gel-forming polymers are polysaccharides.

3. The polymer matrix according to claim 1,
wherein polyvalent cations are the cross-linking agent.

4. The polymer matrix according to claim 1,
wherein the dissolving agent is a complex-forming agent, the complex formation constant of which is higher after activation with the cross-linking agent than the complex formation constant of the binding site.

5. The polymer matrix according to claim 2,
wherein the gel-forming polysaccharide is an alginate.

6. The polymer matrix according to claim 1,
wherein the dissolving agent is present in the cross-linking matrix in a distributed manner.

7. The polymer matrix according to claim 1,
wherein the matrix is a layer system, which is formed from at least two layers, with layers being present with dissolving agent and the layers with dissolving agent being optionally not cross-linked.

8. The polymer matrix according to claim 7,
wherein a non-cross-linked layer with dissolving agent is arranged between cross-linked layers without dissolving agent.

9. The polymer matrix according to claim 1,
wherein the dissolving agent is activated by changing the pH value.

10. The polymer matrix according to claim 1,
wherein the polymer matrix is present in dried form.

11. The polymer matrix according to claim 1,
wherein the polymer matrix contains an incorporated active substance.

12. The polymer matrix according to claim 11,
wherein the active substance is a pharmaceutical.

13. An insert for application on and/or in a human or animal body,
wherein the insert is formed from a polymer matrix according to claim 1.

14. The insert according to claim 13,
wherein the dissolving agent is activated by contact of the polymer matrix with a bodily fluid and the insert dissolves.

15. A use of a polymer matrix according to claim 1 or of an insert according to claim 13 for application on the eye.

16. The use of a polymer matrix according to claim 1 or of an insert according to claim 13 for parenteral application.

17. The use of a polymer matrix according to claim 16 as a resorbable membrane for reducing the formation of postoperative scar tissue in the region of a postoperative site.

18. The use of a polymer matrix according to claim 17,
wherein the membrane is produced in situ.
